(19) United States
(12) Patent Application Publication
(10) Pub. No.: US 2003/0175824 A1
(43) Pub. Date: Sep. 18, 2003

(54) Drug candidate screening systems based on micropatterned hydrogels and microfluidic systems

(75) Inventors: Michael V. Pishko, State College, PA (US); Wong-Gun Koh, State College, PA (US); Alexander Revzin, College Station, TX (US)

Correspondence Address: Paul D. Greeley, Esq., Ohlandt, Greeley, Ruggiero & Perle, L.L.P., 10th Floor, One Landmark Square, Stamford, CT, 06901-2682, US

(73) Assignee: The Penn State Research Foundation

(21) Appl. No.: 10/348,597

(22) Filed: Jan. 21, 2003

Related U.S. Application Data
(60) Provisional application No. 60/351,391, filed on Jan. 22, 2002.

(51) Int. Cl. G01N 33/53; G01N 33/567; C12M 1/34; B05D 3/00

(52) U.S. Cl. 435/7.2; 435/287.2; 427/2.11

(57) ABSTRACT

A cell-containing, three-dimensional hydrogel microstructure that closely imitate a native cell environment. The three-dimensional hydrogel microstructures may be formed using photolithography either alone or in conjunction with the use of microfluidic networks. The resulting cell-containing, three-dimensional hydrogel microstructures can be used efficiently in various cell monitoring applications, including drug candidate screening systems.

### RELATED APPLICATION

The present application claims priority from Provisional Application Serial No. 60/351,391 filed on Jan. 22, 2002.

### FIELD OF THE INVENTION

The present invention relates to micropatterned hydrogels and a method for the encapsulation of cells inside hydrogel microstructures fabricated using photolithography. The present invention also relates to micropatterned hydrogels used with microfluidic systems and a method for fabricating hydrogel microstructures in microfluidic systems.

### BACKGROUND OF THE INVENTION

Cell-based biosensing devices for applications such as high-throughput drug screening require the accurate positioning of cells into arrays that can be addressed (preferably using optical methods) and integrated with microfluidic channels for sample introduction. Much research has been conducted in the area of cell patterning using chemical or lithographic methods for the spatial control of cell adhesion and growth. In most of these applications, anchorage dependent cells are immobilized on a two-dimensional substrate. However, in a two-dimensional system, non-adherent cells are difficult to immobilize and adherent cells, such as fibroblasts and hepatocytes, are in an unnatural environment, i.e., in tissue they exist in a three-dimensional hydrogel matrix consisting of proteins and polysaccharides (i.e., the extracellular matrix). As a result, the response of these cells to drug candidates may be very different than that of the same cells in their native tissue.

Microfluidic devices have gained much attention over the last several years and have significantly influenced the design and the implementation of modern bioanalytical systems. These devices can handle and manipulate small fluid samples in a much more efficient way with the potential of faster assay response times, the simplification of analysis procedures, and smaller samples required for analysis. Microfluidic devices are finding wide applications ranging from synthesis to separations to analysis in applications, such as immunoassays, lab-on-a-chip, rapid nucleotide sequencing, and high throughput screening. Furthermore, microfluidics may be used to pattern biological materials, such as proteins, cells and planar lipid bilayers on substrates with micrometer-scale resolution. Patterned polymer microstructures were also fabricated using microfluidic systems in combination with injection molding. For example, polymer microstructures have been fabricated by molding in capillaries for potential applications in electronic, optical and mechanical devices.

To overcome the problems associated with the prior art two-dimensional culture system, the present invention provides encapsulated cells inside a three-dimensional hydrogel matrix. As a result, a more native three-dimensional cell environment is created resulting in a more efficient screening system.

### SUMMARY OF THE INVENTION

The present invention provides a three-dimensional hydrogel microstructure having cells, bacteria, or both encapsulated therein. The three-dimensional hydrogel microstructure provides a native environment. Therefore, use of the three-dimensional hydrogel microstructures in screening systems results in more efficient screening.

Any suitable cells may be encapsulated in the three-dimensional hydrogel microstructures of the invention. Suitable cells may include, for example, eukaryote, prokaryote, bacterium, or any combinations thereof.

The three-dimensional hydrogel structures of the present invention are formed from one or more polymeric materials. Suitable polymeric materials include, for example, poly(ethylene glycol), poly(2-hydroxyethyl methacrylate), polyvinyl alcohol, hyuronic acid, or any combinations thereof.

To provide a more native environment, the three-dimensional hydrogel structures may also have one or more extracellular matrix components encapsulated with the cells. Suitable matrix components may include, for example, peptides containing integrin binding domains, proteins, polysaccharides, glycoproteins, proteoglycans, or any combinations thereof.

The three-dimensional hydrogel structures of the present invention may be formed in any three-dimensional configuration with any dimensions suitable for encapsulating any number of cells and/or bacteria. By way of example, a suitable height for the hydrogel microstructures is between about 1 μm to about 100 μm and a suitable width is between about 1 μm to about 1000 μm.

The present invention also provides a microfluidic system having one or more three-dimensional hydrogel microstructures, as described above. The microfluidic system can have one or more microchannels formed from one or more polymeric materials. Suitable polymeric materials include, for example, poly(dimethylsiloxane), glass, or silicon.

The present invention also provides a method for forming the one or more three-dimensional hydrogel microstructures described above on a substrate. The method includes the steps of modifying the substrate; applying a suspension to the substrate to form a suspension layer; applying a photomask to the suspension layer wherein portions of the suspension layer are not covered by the photomask; exposing the photomask to UV light, thereby reacting the portions of the suspension layer not covered by the photomask; and removing any unreacted suspension layer from the substrate. As a result, one or more three-dimensional hydrogel microstructures remain on the substrate.

While any substrate may be used, suitable substrates include, for example, glass, silicon, plastic, rubber, ceramics, or any combinations thereof. The substrate is modified with a component to promote good adhesion. Suitable components for modifying the substrate include, for example, alkoxysilanes, halosilanes, alkyl thiols, alkylphosphonates, or any combinations thereof.

A cell-containing polymer suspension is used to form the hydrogel microstructures. The suspension may include any suitable components for forming the microstructures. Suitable components include, for example, poly(ethylene glycol), poly(ethylene glycol) diacrylate, poly(ethylene glycol) dimethacrylate, photoinitiator, cell suspension, cell culture media, cell adhesion molecules such as collagen or fibronectin, cell adhesion peptides, polysaccharides, glycoproteins, proteoglycans, or any combinations thereof.

The suspension can be applied to the substrate by any suitable method for forming a suspension layer on the substrate. Suitable methods include, for example, spin-coating, pin printing, or microreaction injection molding. When spin-coating is used to apply the suspension to the substrate, spin-coat rates can range, for example, between about 1000 rpm to about 5000 rpm.

The present invention also provides a method of forming one or more three-dimensional hydrogel microstructures on a substrate with a microfluidic network. The method includes the steps of forming a microfluidic network having one or more microchannels on the substrate; filling the one or more microchannels with a gel precursor solution; exposing the gel precursor to UV light; and removing the microfluidic network from the substrate. As a result, one or more molded three-dimensional hydrogel microstructures remain on the substrate.

It is also within the scope of the invention to provide micropatterned three-dimensional hydrogel microstructures using a microfluidic network. To achieve micropatterns, prior to exposing the gel precursor solution to UV light, a photomask of any desired pattern can be placed over the one or more microchannels. As a result, a predetermined pattern is exposed in the gel precursor, resulting in micropatterned three-dimensional hydrogel microstructures.

The present invention also provides a method of analyzing one or more cells by forming one or more three-dimensional hydrogel microstructures having one or more cells to be analyzed encapsulated therein; and analyzing the one or more cells.

While any analyzation means may be used, the one or more cells can be analyzed by a monitoring means including, for example, fluorescence including lifetime and polarization techniques, electrochemical, absorbance, chemiluminescence, surface acoustic wave mass sensors, magnetoelastic mass sensors or any combinations thereof. The one or more cells can be analyzed for one or more effects including, for example, toxicity, cell morphology, apoptosis, differentiation, cell-cell interaction, cell-matrix interaction, host-pathogen interaction, endocytosis, exocytosis, or any combinations thereof.

The present invention also provides a method for drug candidate screening. The method includes the steps of preparing a substrate having one or more cell-containing three-dimensional hydrogel microstructures disposed thereon; delivering one or more reagents to the one or more cell-containing three-dimensional hydrogel microstructures; contacting the one or more reagents with one or more cells encapsulated in the one or more cell-containing three-dimensional hydrogel microstructures; and monitoring the one or more cells.

Suitable reagents for use in the present invention may include, for example, pharmaceutical drug candidates or unknown sample containing potentially bioactive compounds. The reagents may be delivered to the microstructures by any suitable means, which include, for example, pressure-driven flow, capillary flow, electro-osmotic flow, or any combinations thereof.

The one or more cells can be monitored by any suitable means including, for example, fluorescence including lifetime and polarization techniques, electrochemical, absorbance, chemiluminescence, surface acoustic wave mass sensors, magnetoelastic mass sensors or any combinations thereof. The one or more cells can be monitored for one or more effects including, for example, toxicity, cell morphology, apoptosis, differentiation, cell-cell interaction, cell-matrix interaction, host-pathogen interaction, endocytosis, exocytosis, or any combinations thereof.

### DESCRIPTION OF THE DRAWINGS

FIG. 1 is a micrograph of hydrogel microstructures on a flexible silicone rubber substrate according to the present invention;

FIG. 2 is an optical transmission micrograph of mouse 3T3 fibroblasts spreading in fibronectin modified microstructures according to the present invention;

FIG. 3 is an ESEM micrograph of 50 μm diameter hydrogel microstructures containing 3T3 fibroblast according to the present invention;

FIG. 4 is a chart showing the reproducible encapsulation of cells within 100×100×100 micrometer hydrogel microstructures having mouse 3T3 fibroblasts according to the present invention;

FIG. 5(a) is a micrograph showing a cell-containing hydrogel precursor solution in a microchannel according to the present invention;

FIG. 5(b) is a micrograph showing the gelation of the hydrogel inside the microchannel after exposure to UV light through a photomask according to the present invention;

FIG. 5(c) is a micrograph showing a cell-containing hydrogel microstructure inside a microchannel after the removal of unreacted precursor solution according to the present invention;

FIG. 6 shows a schematic diagram of the photoreaction injection molding process for the fabrication of hydrogel microstructures according to the present invention;

FIG. 7 is a micrograph of a hydrogel microstructure in the shape of a microchannel on a glass substrate according to the present invention;

FIGS. 8(a)-(d) are micrographs of cylindrical hydrogel microstructures during fabrication inside a microchannel according to the present invention;

FIGS. 9(a) and (b) are micrographs of microstructures with 6 channels during fabrication according to the present invention;

FIGS. 10(a) and (b) are micrographs of the microstructures of FIG. 9 after removing the PDMS template and washing away unreacted precursor solution according to the present invention; and

FIGS. 11(a)-(c) are micrographs of a heterogeneous hydrogel microstructure according to the present invention, visualized with bright field and fluorescence microscopy.

### DETAILED DESCRIPTION OF THE INVENTION

High-density arrays of three-dimensional microstructures are created on substrates using photolithography. Fabrication of these arrays involves immobilizing either single or small groups of cells and/or bacteria in three-dimensional poly(ethylene glycol) hydrogel microstructures fabricated on plastic or glass surfaces. These hydrogel microstructures are then engineered to contain adhesion peptides, proteins, and any other suitable extracellular matrix components to create an environment as close to that of native tissue as possible. Immobilizing cells within a three-dimensional microstructure more closely mimics the native three-dimensional environment of a cell than does cells cultured on a planar substrate, such as tissue culture polystyrene. The cell-containing microstructures can then be integrated with microfluidic systems designed to supply media and introduce drug candidates to the cellular array. The response of these cells to the candidates may be monitored using any known monitoring means, such as, for example, fluorescent reporters and/or electrochemical detectors and analyzed to quantify the effect of these agents on the different phenotypes present in the array. Besides non-specific effects, such as toxicity, parameters, such as, for example, cell morphology, apoptosis, differentiation, cell-cell interactions (same phenotype and between phenotypes), and cell-matrix interactions may be quantified.

The present invention is unique in that it allows for the creation of micropatterned three-dimensional hydrogel structures encapsulating viable mammalian cells on glass, silicon and plastic, including, but not limited to, flexible substrates. It enables the patterning of multiple phenotypes on a single platform and the creation of hydrogel microstructures with an interface of cells of differing phenotype (e.g., a gel microstructure with a region of endothelial cells adjacent to a region of hepatocytes). Cell adhesion molecules can be integrated in the hydrogel structure while the permeability, charge, and equilibrium water content of the hydrogel can all be controlled. Laminate hydrogel microstructures can be created and cell-containing microstructures and microfluidic channels can be fabricated in one step. Three-dimensional cell containing structures can be fabricated over microelectrodes (both amperometric and potentiometeric). Simultaneous fluorescent and electrochemical sensing can be performed on encapsulated cells. Cell-containing microstructures can be fabricated and “floated” into position elsewhere in a microfluidic system. Finally, the invention enables the fabrication of gel-based filters and chromatography features in microfluidic channels.

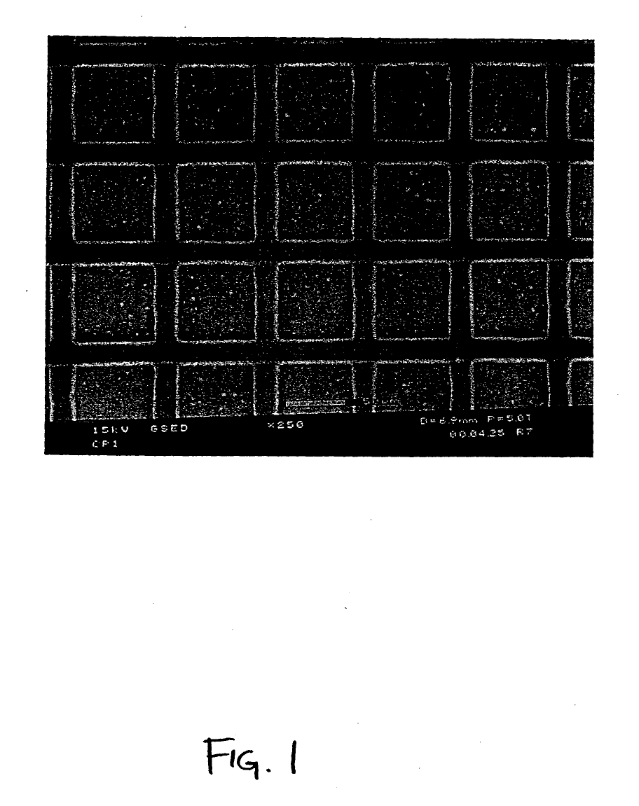

In one embodiment of the present invention, the fabrication, using photolithography of poly (ethylene glycol) (PEG)-based hydrogel microstructures encapsulating viable mammalian cells on glass and silicon substrates is described. Mammalian cells were encapsulated in cylindrical hydrogel microstructures of 600 and 50 μm in diameter or in cubic hydrogel structures in microfluidic channels. Reducing lateral dimension of the individual hydrogel microstructure of 50 μm allowed 1 to 3 cells per microstructure to be isolated. Viability assays demonstrated that cells remained viable inside these hydrogels after encapsulation for up to seven days. By way of example, FIG. 1 depicts hydrogel structures formed on a flexible silicone rubber substrate according to the present invention.

The following is a preferred embodiment of the present invention and is in no way intended to limit the scope of the invention. To form cell-containing hydrogel microstructures on surfaces, the surfaces of glass and silicon substrates are modified to promote good adhesion, essential for the gel to remain stationary in a flow field. The substrate surface is modified with an organosilane to create surface-tethered methacrylate groups capable of covalent bonding with hydrogel during photopolymerization. By way of example, substrates are first immersed in ‘piranha’ solution consisting of a 3:1 ratio of 30% w/v H₂O₂ and H₂SO₄ to clean and hydroxylate the substrate surface. The hydroxylated surface was then immersed for 5 minutes in a 1 mM solution of 3-(trichlorosilyl)propyl methacrylate (TPM, Sigma-Aldrich) in 80%/20% (v/v) mixture of heptane/carbon tetrachloride, which resulted in the formation of a dense network of Si—O—Si bonds on the substrate surface and pendant methacrylate functionalities at the substrate/solution interface as confirmed by TOF-SIMS. This surface modification was easily visualized by the increase in water contact angle associated with hydrophobic methacrylated alkylsilanes on hydrophilic SiO₂. Ellipsometry measurements of modified Si/SiO₂ surfaces indicated that the organosilane films were 14±3 Å thick, indicating the presence of a monolayer of TPM on the substrate surface.

Hydrogel microstructures encapsulating murine 3T3 fibroblasts were fabricated using proximity photolithography. A PEG-diacrylate (PEG-DA, MW 575, Sigma-Aldrich or MW 4000, Polysciences) precursor solution containing 0.5% (w/w) DAROCUR 1173 (1-phenyl-2-hydroxy-2-methyl-1-propanone, Ciba Specialty Chemicals) as a photoinitiator was mixed with a cell suspension in cell culture media to produce a cell density about 4 to 5×10⁶ cells/mL in the gel precursor solution. Fibroblasts were cultured on tissue culture polystyrene in Dulbecco's modified Eagle media (DMEM with 4.5 g/L glucose and 10% FBS, Sigma-Aldrich) and incubated at 37° C. in 5% CO₂ and 95% air until near confluence. Cells were detached from culture flasks by trypsinization with 0.25% trypsin and 0.13% EDTA in phosphate buffered saline. Cells were transferred back to cell culture media and then added to the gel precursor solution. The cell-containing polymer suspension was spin-coated onto functionalized substrates at 1500 rpm for 10 seconds to form uniform fluid layer. This layer was covered with a photomask and exposed to 365 nm UV light (300 mW/cm²) for 0.5 seconds through the photomask. Upon exposure to UV light, only exposed regions underwent free-radical induced gelation and became insoluble in common PEG solvents such as water. As a result, desired microstructures were obtained by washing away unreacted precursor solution with phosphate buffered saline (PBS) or cell culture medium so that only the hydrogel microstructures remained on the substrate surface. During the UV light induced gelation process, cells suspended in the polymer precursor solution were encapsulated in the resultant hydrogel microstructures. Serum proteins present in the precursor solution were also likely entrapped in the gel to some extent. After encapsulation, surfaces with cell-containing microstructures were immersed in cell culture media (DMEM with 10% fetal bovine serum) and incubated in a 5% CO₂ atmosphere at 37° C. to assess viability.

Methacrylate moieties on the substrate surface also participate in the free radical polymerization and create covalent bonding between acrylate groups present in the bulk gel and those on the surface, thus fixing the hydrogel structures to the substrate. Long term adhesion of cell-containing hydrogel arrays to silicon surface was verified by placing hydrogel elements into an aqueous environment for over a week at ambient temperature. Upon hydration, PEG hydrogels may expand in volume by over 100%. In the absence of covalent attachment to the substrate, the mechanical forces associated with swelling are sufficient to cause the gels to delaminate from the surface. Here, the TPM monolayer binds the gel to the surface and prevents delamination while still allowing the gel to swell with aqueous media. However, the bound gel tends to swell anisotropically, i.e., the dimensions at the base of the gel do not change but rather the gel swells upward away from the surface. The gels of the present invention were fabricated at approximately their equilibrium water content because of the aqueous cell culture media added along with the cells. Thus, the gels do not physically swell with additional water. However, covalent attachment of the gels to the substrate surface is still necessary as unattached gels are easily washed from the surface.

To optimize the size of the cell-containing microstructures, various spin-coating rates were tested to create thicker gels and microstructures with greater aspect ratios. As expected, the thickness of the deposited layer of precursor solution was found to be inversely proportional to the spin-rate, and thus allowed control over the height of hydrogel microstructures. Spin-rates of 4000 rpm resulted in cylindrical hydrogels of about 10 μm in height as measured by profilometry, while polymer layer spun at 1500 rpm yielded hydrogel elements about 70 μm in height as observed by environmental scanning electron microscopy (ESEM). Hence, both lateral and vertical dimensions of hydrogel microstructures can be controlled, the former by feature size of the photomask to a minimum size of 7 μm and the latter by the spin-coating rate. By using masks with different feature sizes and using different spin-coating rates, it is possible to create cell-containing microstructures with aspect ratios ranging from about 0.12 to about 1.4.

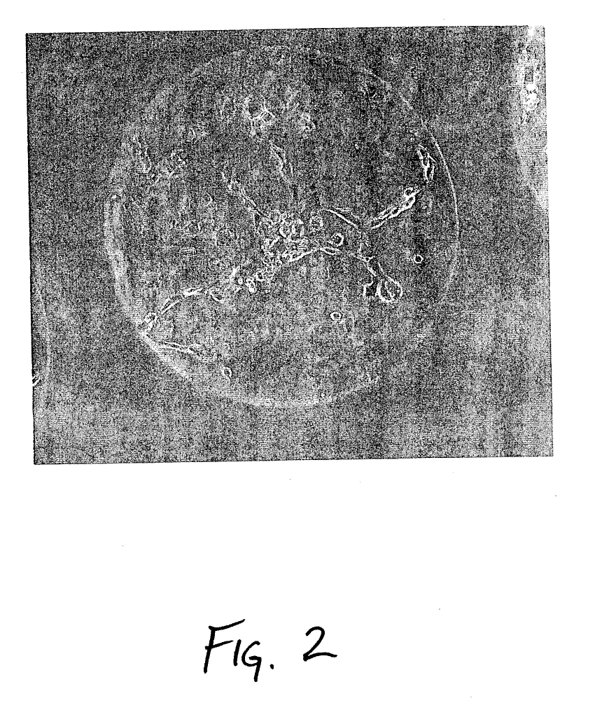

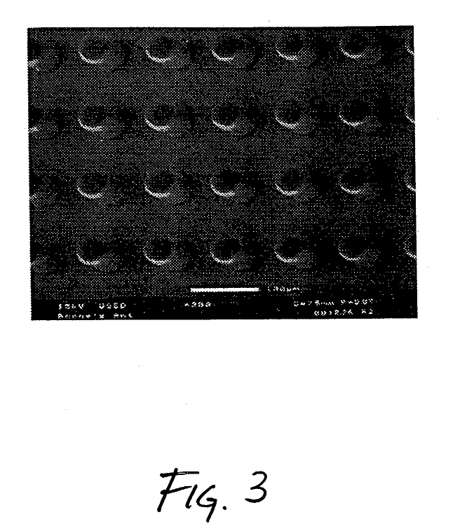

FIG. 2 shows the optical transmission micrograph of a hydrogel microstructure containing mouse 3T3 fibroblasts. The cells were completely encapsulated within the microstructures with no cells or cell processes evident outside the gel. The transparent nature of PEG-based hydrogel allows for the observation of cells in the hydrogel structure through optical microscopy without staining. An approximately equal number of cells (30 per microstructure) are observed in each of the several hydrogel elements. Even though the size resolution of proximity lithography is larger than that of contact lithography, high-quality hydrogel microstructures of 50 μm diameter are obtained, as shown by the electron micrograph in FIG. 3. These cylindrical microstructures are of a three-dimensional nature and are arranged in a 20×20 square with 50 μm spacing between elements so that as many as 400 microstructures can be reproducibly fabricated in a 2 mm² area. While 600 μm hydrogel microstructures contained numerous cells, 50 μm diameter microstructures have only 1 to 3 cells encapsulated per structure, with some microstructures absent of cells. In both types of microstructures, encapsulated cells appear rounded even after 24 hours but were found to spread slowly over the course of several days. The slow rate of spreading by encapsulated cells is likely caused by insufficient protein in the gel, as PEG inhibits cell adhesion and proteins, such as collagen, are required for cell adhesion and spreading. Thus, cells may not have spread until they themselves produced sufficient extracellular matrix.

As is apparent from FIG. 3, the fact that these microstructures contain cells is not readily evident by electron microscopy because the cells are completely encapsulated within the gel. Indeed, even if cells were in the microstructures, the question remains as to whether they are viable or not. Cell viability was anticipated because UV (ultraviolet) polymerization conditions and chemical components were chosen to minimize cytotoxicity and the resulting gels possessed sufficient permeability to permit the transport of nutrients and oxygen to the cells. The viability of individual cells in hydrogel microstructures with diameters of 600 μm and 50 μm was investigated using Live/Dead Viability/Cytotoxicity fluorescence assay (Molecular Probes, Inc.) that stains live cells green and dead cells red. By using this type of assay, cells within microstructures could be imaged and assayed for viability simultaneously. It was found that approximately 80% of the encapsulated cells were viable, demonstrating that the conditions for fabrication were sufficient for encapsulating viable cells in the photopolymerized PEG hydrogel. Cells encapsulated in PEG microstructures based on MW 575 PEG-DA lost viability after 3 days while those based on MW 4000 PEG-DA remained viable for 7 days. Murine SV-40 transformed hepatocytes were also encapsulated in a similar fashion and also retained viability.

As a control, cell-containing microstructures were incubated with 0.05% sodium azide in PBS. Azide anion killed the cells in the microstructures as anticipated and resulted in cells that stained red in the LIVE/DEAD assay. The viability of cells encapsulated in hydrogel was also measured using an MTT (3(4,5-dimethylthiazol-2-yl)-2,5-diphenyl tetrazolium bromide) assay. In this assay, viable cells generated purple formazan crystals and confirmed that cells within the microstructures were viable.

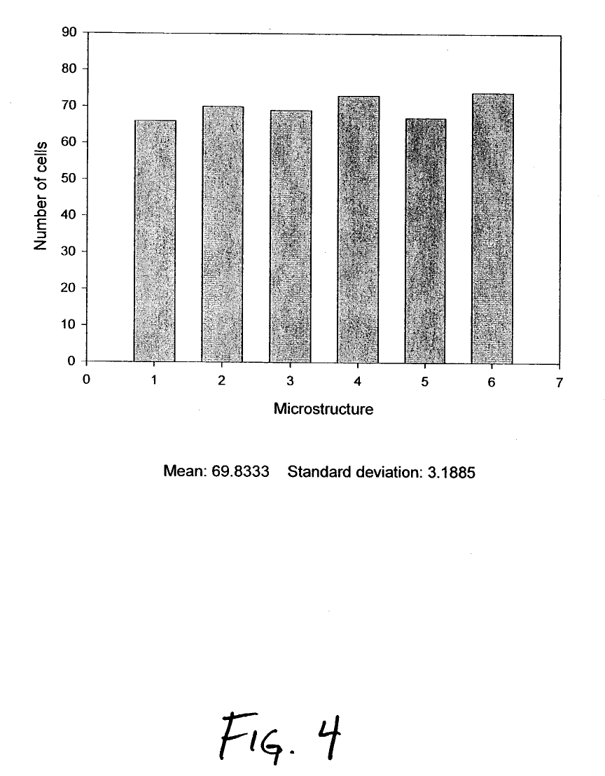

With the present invention, encapsulation of viable cells within the hydrogel microstructures is highly reproducible, as evident by FIG. 4. As can been seen, virtually the same number of cells appear in all six microstructures examined.

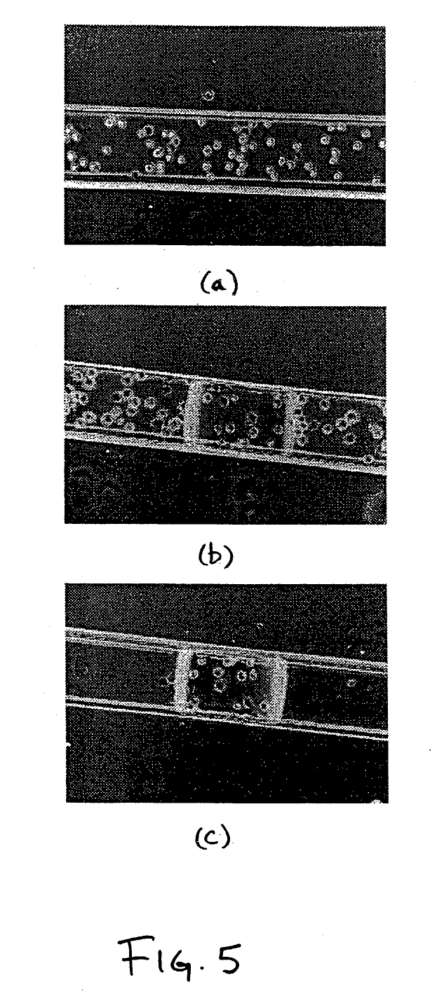

Based on these results, cell-containing hydrogel microstructures were prepared inside microfluidic channels. By way of example, an approximately 100 μm wide, 50 μm deep microchannel was created in poly(dimethyl siloxane), treated in an O₂ plasma to improve adhesion, and was sealed irreversibly to a glass slide to form an enclosed microchannel. This microchannel was filled with a cell-containing hydrogel precursor solution (FIG. 5(a)) and then exposed to UV light through a photomask. Only illuminated regions underwent photopolymerization and gelled inside the microchannel as shown in FIG. 5(b). Finally, by flushing the channel with PBS, it was possible to obtain the desired cell-containing hydrogel microstructure inside a microfluidic channel as shown in FIG. 5(c).

Cell viability and function with these gel microstructures and the formulation of gel chemistries designed to improve cell proliferation and function, perhaps through the inclusion of cell adhesion molecules such as collagen, fibronectin, vitronectin or their peptide analogs may be determined by employing the microstructures described herein. In addition, the microstructures may be combined with a microfluidic device to create optical biosensor arrays of individually addressable single or multiple cell-containing hydrogel microstructures for application in drug screening or pathogen detection.

The present invention also provides for reaction injection molding using in situ photoinduced polymer macromer gelation in microfluidic channels applied to the fabrication of poly(ethylene glycol) (PEG) hydrogel microstructures. These hydrogel microstructures are fabricated using poly(dimethylsiloxane) (PDMS) microchannels as mold inserts alone or in combination with photolithography. These microstructures are formed by flowing a gel precursor solution through the microfluidic network, exposing it to light, and finally removing the PDMS mold. Microchannels as narrow as 10 μm wide can be used for molding PEG hydrogels and the resulting three dimensional hydrogel microstructures do not delaminate from substrates treated with a gel adhesion promoter, such as, for example, 3-(trichlorosilyl)propyl methacrylate (TPM). By exploiting the laminar flow and poor mixing conditions in a microfluidic channel, single microstructures with heterogeneous chemistries are also created, using peptide-modified structures to promote cell adhesion.

Here we describe the fabrication of the PEG hydrogel microstructures via photoreaction injection molding using microfluidic networks of PDMS as micromolds. Acrylate or methacrylate modified macromers are injected into the fluidic system and light is used to gel the macromers to form a hydrogel. The PDMS replica is then removed, leaving the gelled microstructures on the substrate surface. By combining photoreaction injection molding with photolithography, arrays of hydrogel microstructures possessing different chemistries are created. By exploiting the slow diffusion driven mixing that occurs in microfluidic channels, microstructures with heterogeneous chemical structures are created. Microstructures that possessed cell adhesion molecules on one portion of the microstructure and lacked them on another were fabricated as a model system.

By way of example, the fabrication of PEG hydrogel microstructures via photoreaction injection molding using microfluidic networks is described below.

Preparation of microfluidic networks

Microfluidic networks are formed from a 10:1 mixture of the PDMS prepolymer and the curing agent. The resulting mixture was poured on the silicon masters and cured at 60° C. for at least 2 hours. The silicon masters have a negative pattern of the desired micropattern defined with SU-8 50 negative photoresist (Microlithography Chemical Corp., Newton, Mass.). After curing, the PDMS replica was removed from the master and treated in an oxygen plasma (Harrick Scientific Co., Ossining, N.Y.) for 1 minute to change its hydrophobic surface to hydrophilic. Glass substrates were modified with a 3-(trichlorosilyl)propyl methacrylate (TPM) monolayer to enhance the adhesion of hydrogel microstructures to glass surfaces. The oxidized microfluidic networks were placed by hand on the TPM-modified glass to form an enclosed channel and pierced from the backside of the network with syringe needles to open a path for incoming fluids. These PDMS microchannel systems were used as mold inserts for photoreaction injection molding.

Reaction injection molding using photopolymerization

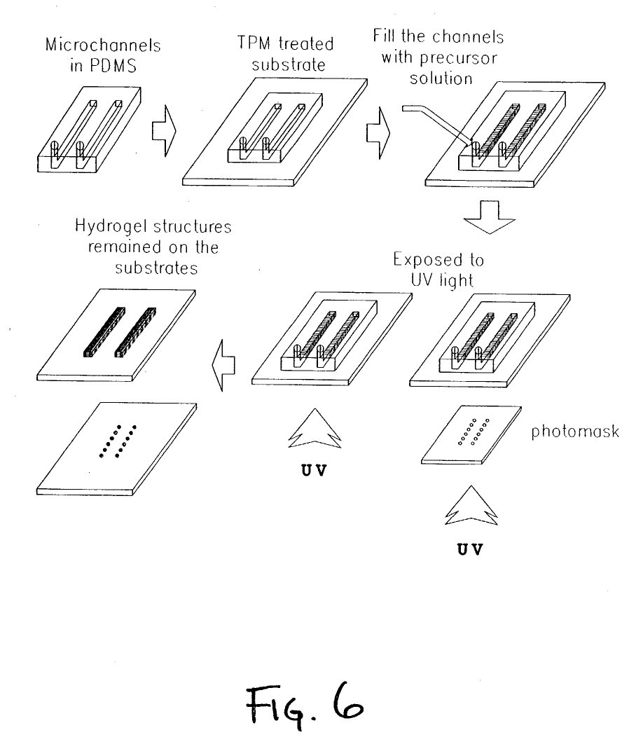

Hydrogel microstructures were fabricated using PEG-DA (MW 575 or 4000) macromers. The gel precursor solution was composed of 20% w/v of PEG DA and 0.1% w/v of photoinitiator in cell culture medium or PBS. To create these hydrogel microstructures, each independent microchannel was filled with gel precursor solution and then exposed to 365 nm, 300 mW/cm² UV light (EFOS Ultracure 100 ss Plus, UV spot lamp, Mississauga, Ontario) for 1 second. To make cylindrical hydrogel microstructures within a microfluidic channel, photomasks possessing the desired design were aligned over the microchannels and exposed to light. The precursor solution exposed to UV light undergoes free-radical cross-linking and becomes insoluble in common PEG solvents, such as water. After the precursor solution gels, the PDMS microfluidic network was quickly removed from the glass substrate to obtain the molded hydrogel microstructures. FIG. 6 shows a schematic diagram of the photoreaction injection molding process, both with and without using a photomask, for the fabrication of hydrogel micro structures.

Cell culture

Murine fibroblasts were cultured in DMEM with 4.5 g/L glucose and 10% FBS and are incubated at 37° C. in 5% CO₂ and 95% air. Fibroblasts were grown to confluence in 75 cm² polystyrene tissue culture flasks and confluent cells are subcultured every 2 to 3 days by trypsinization with 0.25% (w/v) trypsin and 0.13% (w/v) EDTA.

Formation of PEG hydrogel using photoreaction injection molding

The fabrication system for photoreaction injection molding consists of two parts. The first part is a microstructured mold insert formed from PDMS and the second is a TPM-modified glass substrate. These two parts were sealed together to form the complete mold and are subsequently filled with hydrogel precursor solution. PDMS microfluidic networks were fabricated by replica molding, which creates a PDMS replica possessing three of the four walls necessary for the enclosed microfluidic channels. The angle of the walls was almost 90 degrees, so the microchannel in the PDMS replica was essentially rectangular. The depth of the microchannel is fixed to about 50 μm and the width is either 200 or 300 μm. Sealing the replica to a flat glass surface creates a complete microchannel network. Reversible, conformal sealing with TPM-modified glass surfaces is used. Reversible sealing between the PDMS replica and glass occurs due to the softness of PDMS and its ability to conform to minor imperfections in a flat surface, thus making van der Waals contact with these surfaces. PDMS microchannels were easily peeled off from the glass substrate with only moderate force and without leaving significant PDMS residue on the substrate. Therefore, resealing of the replica to the substrate can be performed numerous times with the same PDMS replica.

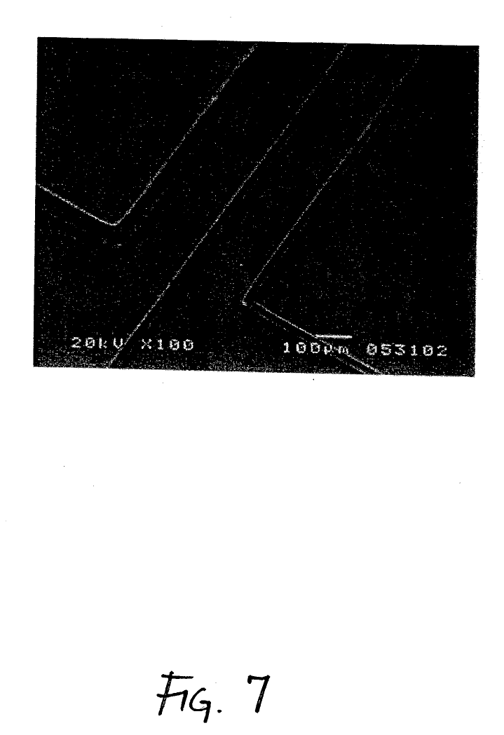

For the photoreaction injection molding of PEG hydrogels, the gel precursor solution must completely fill the microchannels. Since reversible sealing cannot withstand high pressure in the microchannels, the precursor solution should fill the channel by either capillary action or via pressure-driven flow at a low flow rate. For the precursor solutions described here, however, both PDMS and TPM-modified glass surfaces are hydrophobic; therefore, the solution cannot flow through the channel by capillary action. To solve this problem, PDMS microchannels are treated with an oxygen plasma to make them hydrophilic. Oxygen plasma treatment lowers the contact angle of channel surfaces with water to almost zero, allowing channels to be easily filled with the gel precursor solution via capillary action. After the filled channels are exposed to UV light for 1 second, the PDMS replica is removed from the glass substrate. Hydrogel microstructures remain on the TPM-modified substrates after removal of PDMS microchannels and do not detached from the substrate when exposed to an aqueous environment for a week because of the covalent bonding between the hydrogel microstructures and the surface-tethered methacrylate groups on the substrate. FIG. 7 shows the resultant replicated hydrogel microstructures, which assumed the shape of the microchannels, remained on the glass substrates. Clearly defined three-dimensional hydrogels were fabricated with smooth surfaces and as narrow as a 10 μm-wide microchannel can be used for the fabrication of hydrogel microstructures.

Fabrication of arrays of hydrogel microstructures

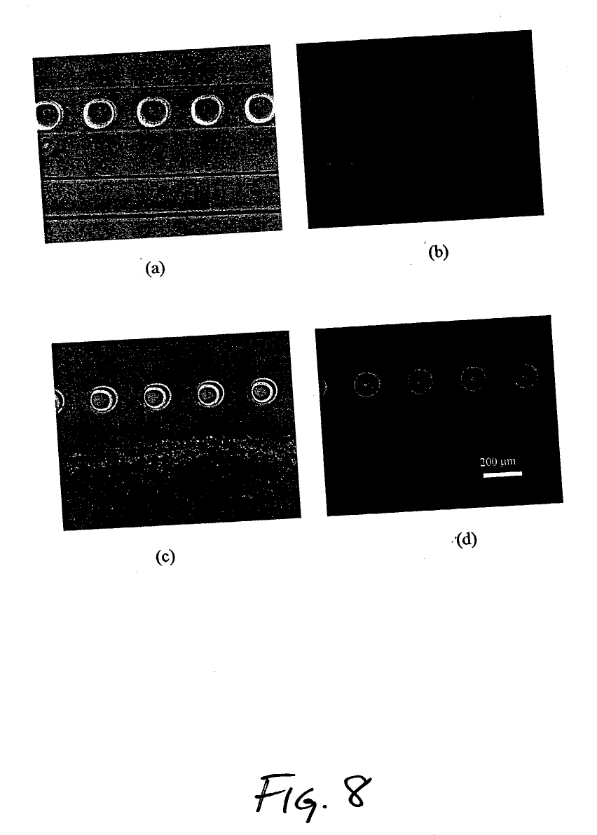

More complicated hydrogel microstructures are produced by combining photolithography with photoreaction injection molding. Examples are shown in FIG. 3. Here, after a gel precursor solution containing fluorescein is injected to microchannels, a photomask with the design of 100 μm diameter circular array is aligned with the channels and exposed to UV light. As shown in FIGS. 8(a) and 8(b), the resulting cylindrical hydrogel microstructures are fabricated inside the microchannel in the UV-illuminated regions while unpolymerized gel precursor solution remained in the microchannel in the unexposed region. By removing PDMS and rinsing the glass slide with water, the desired cylindrical elements of PEG hydrogel are obtained, as shown FIGS. 8(c) and 8(d).

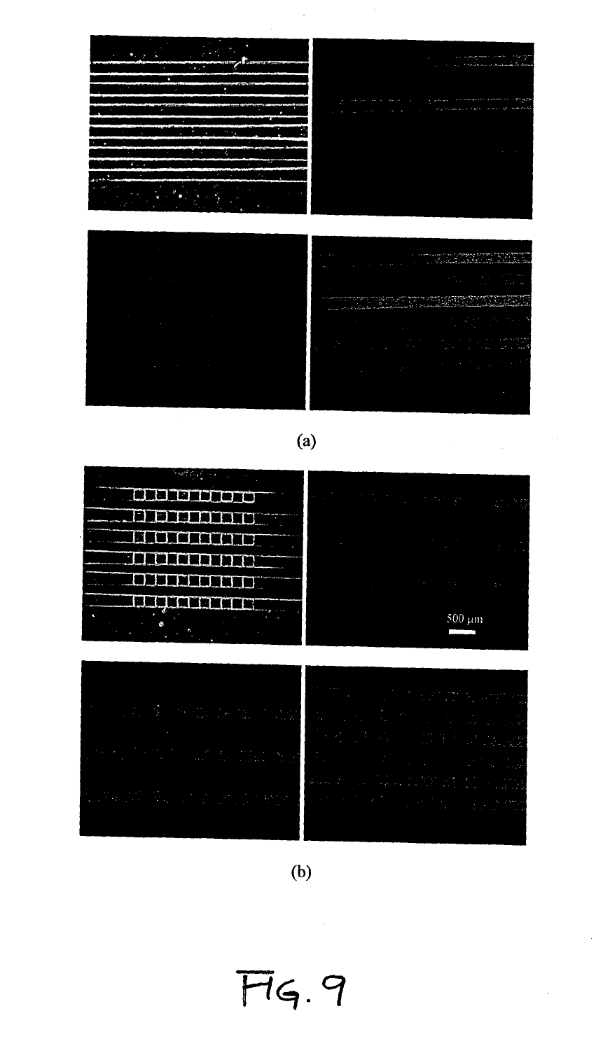

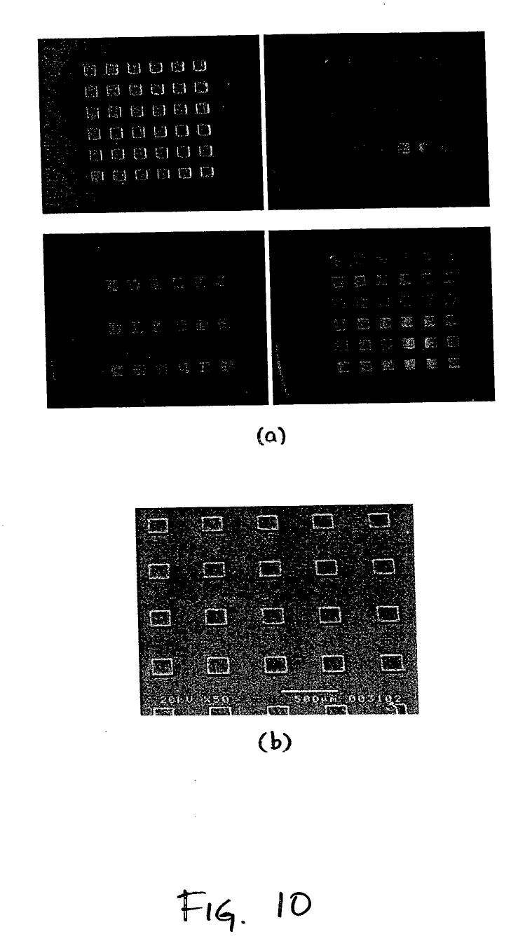

By using the photoreaction injection molding technique of the present invention, clear advantages were discovered over previous methods in fabricating hydrogel microstructures. For example, only a small volume of precursor solution is needed to fill a microchannel. Another important advantage is that hydrogel microstructures possessing different chemistries can be easily fabricated on a single substrate without the need for multiple spin-coating, alignment, exposure and developing steps, as with conventional photolithography. Because sets of microchannels are fluidically isolated from each other, the simultaneous introduction of independent gel chemistries into each channel is permitted and microstructures can be created using only a single photolithographic exposure. Referring to FIG. 9, to fabricate microstructures in this fashion, a mold insert composed of six channels is first fabricated in PDMS. Gel precursor solutions including fluorescein and tetramethylrhodamine are alternatively introduced to each microchannel (FIG. 9(a)). These precursor solution-containing microchannels are then exposed to UV light through a photomask (FIG. 9(b)). Removing the PDMS template and washing away the unreacted precursor solution with water results in an array of hydrogel microstructures that contain both fluorescein and tetramethylrhodamine, as shown in FIG. 10(a). By using this technique, multiple cell phenotypes and proteins can be created in an array of hydrogel microstructures with a lower probability of chemical cross-contamination between structures than one would see with multiple spin-coating procedures, as seen in FIG. 10(b).

Patterning heterogeneous hydrogel microstructures inside microchannels

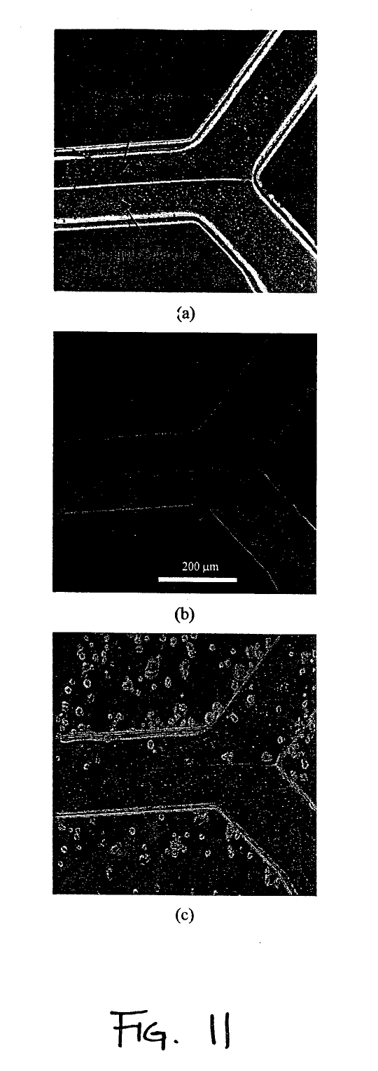

An important characteristic of flow inside microfluidic channels is that the flow has a low Reynolds number and is laminar. When two or more streams with low Reynolds number are introduced to a single microchannel simultaneously, the combined streams flow parallel to each other with mixing between the streams occurring only by diffusion. Using this flow property inside a microchannel, two precursor solutions with different chemistries are introduced to a Y-shaped microchannel using a syringe pump (Harvard Apparatus, Holliston, Mass.). One precursor solution containing PEG-DA, initiator and tetramethylrhodamine is introduced on one branch of the microchannel while the other precursor solution containing RGD peptides in addition to PEG-DA and initiator is introduced in the other branch. The peptides are conjugated to the hydrogel network by reacting the peptides with acryloyl-PEG-N-hydroxysuccinimide (acryloyl-PEG-NHS, 3400 Da; Shearwater Polymers, Huntsville, Ala.). As the two solutions are united in the microfluidic system, they remain distinct and do not visibly mix. Photogelation of the two precursor solutions is performed and then the PDMS microfluidic mold is removed to obtain the final hydrogel microstructures. FIGS. 11(a) and 11(b) show the resultant heterogeneous hydrogel microstructure visualized with bright field and fluorescence microscopy. As shown in these images, a hydrogel microstructure having a polarized chemistry is fabricated inside the microchannel as is clear from the interface between the two regions shown in FIG. 11(a) and the fluorescence image in FIG. 11(b). To demonstrate that the two regions of the hydrogel microstructure are functionally distinct, 3T3 murine fibroblasts are seeded on the patterned substrate and attached cells are observed after 10 hour incubation. Because of the extremely hydrophilic nature of PEG, cells are unable to adhere to the region of the microstructure that does not have the RGD adhesion peptide, whereas cell adhesion improved dramatically on the surface of the region that incorporated RGD, as shown in FIG. 11(c). The creation of hydrogel microstructures that show such differences in cell adhesion allows one to create novel biomaterial microstructures to promote the development of microstructured tissue.

It should be understood that the foregoing description is only illustrative of the present invention. Various alternatives and modifications can be devised by those skilled in the art without departing from the invention. Accordingly, the present invention is intended to embrace all such alternatives, modifications and variances.

What is claimed is:

1. A three-dimensional hydrogel microstructure having encapsulated therein at least one cell.

2. The three-dimensional hydrogel microstructure of claim 1, wherein said cell is a eukaryote, prokaryote, or mixture thereof.

3. The three-dimensional hydrogel microstructure of claim 1, wherein said hydrogel microstructure is formed from at least one polymeric material.

4. The three-dimensional hydrogel microstructure of claim 2, wherein said polymeric material is poly(ethylene glycol).

5. The three-dimensional hydrogel microstructure of claim 1, further comprising at least one extracellular matrix component encapsulated within said hydrogel microstructure.

6. The three-dimensional hydrogel microstructure of claim 5, wherein said extracellular matrix is at least one selected from the group consisting of: peptides, proteins, polysaccharides, glycoproteins, proteoglycans, and any combinations thereof.

7. The three-dimensional hydrogel microstructure of claim 1, wherein said cell is a mammalian cell.

8. The three-dimensional hydrogel microstructure of claim 1, wherein said encapsulated cells are comprised of two or more phenotypes.

9. The three-dimensional hydrogel microstructure of claim 1, wherein said hydrogel microstructure has a height between about 1 μm to about 100 μm.

10. The three-dimensional hydrogel microstructure of claim 1, wherein said hydrogel microstructure has an aspect ratio between about 0.12 to about 1.4.

11. A microfluidic system comprising at least one three-dimensional hydrogel microstructure.

12. The microfluidic system of claim 11, further comprising at least one microchannel.

13. The microfluidic system of claim 12, wherein said microchannel is formed from at least one polymeric material.

14. The microfluidic system of claim 12, wherein said microchannel is formed from poly(dimethylsiloxane).

15. The microfluidic system of claim 12, wherein said microchannel is formed in glass.

16. The microfluidic system of claim 12, wherein said microchannel is formed in silicon

17. The microfluidic system of claim 1 1, wherein said three-dimensional hydrogel microstructure is formed from at least one polymeric material.

18. The microfluidic system of claim 17, wherein said polymeric material is poly(ethylene glycol).

19. The microfluidic system of claim 1 1, wherein said three-dimensional hydrogel microstructure has at least one cell encapsulated within said three-dimensional hydrogel.

20. The microfluidic system of claim 19, wherein said at least one cell is an eukaryotic cell.

21. The microfluidic system of claim 19, wherein said at least one cell is a mammalian cell.

22. The microfluidic system of claim 19, wherein said at least one cell is a prokaryotic cell.

23. The microfluidic system of claim 19, wherein said at least one cell is a bacterium.

24. The microfluidic system of claim 19, wherein said at least one cell is at least two mammalian cells of two or more phenotypes.

25. The microfluidic system of claim 19, wherein said at least one cell is at least one mammalian cell and bacteria.

26. The microfluidic system of claim 11, wherein said three-dimensional hydrogel microstructure also has at least one extracellular matrix encapsulated therein.

27. The microfluidic system of claim 26, wherein said at least one extracellular matrix is selected from the group consisting of: peptides, proteins, polysaccharides, glycoproteins, proteoglycans, and any combinations thereof.

28. The microfluidic system of claim 11, wherein said three-dimensional hydrogel microstructure has a height between about 1 μm to about 100 μm.

29. The microfluidic system of claim 11, wherein said three-dimensional hydrogel microstructure has an aspect ratio between about 0.12 to about 1.4.

30. A method of forming a three-dimensional hydrogel microstructure on a substrate, said method comprising:
applying a suspension to said substrate to form a suspension layer;
applying a photomask to said suspension layer wherein portions of said suspension layer are not covered by said photomask;
exposing said photomask to an ultraviolet light source, whereby said portions of said suspension layer not covered by said photomask are reacted; and
removing any unreacted suspension layer from said substrate,
wherein said three-dimensional hydrogel microstructure is formed on said substrate.

31. The method of claim 30, wherein said substrate is selected from the group consisting of: glass, silicon, plastic, rubber, ceramic, and any combinations thereof.

32. The method of claim 30, further comprising the step of modifying said substrate prior to applying said suspension, wherein said substrate is modified with at least one component selected from the group consisting of: alkoxysilanes, halosilanes, alkyl thiols, alkyl phosphonates, and any combinations thereof.

33. The method of claim 30, wherein said suspension comprises at least one component selected from the group consisting of: poly(ethylene glycol), poly(ethylene glycol) diacrylate, poly(ethylene glycol) dimethacrylate, photoinitiator, cell suspension, cell culture media, cell adhesion molecules, collagen, fibronectin, cell adhesion peptides, polysaccharides, glycoproteins, proteoglycans, and any combinations thereof.

34. The method of claim 30, wherein said suspension is applied to said substrate by spin-coating.

35. The method of claim 30, wherein said suspension is applied to said substrate by flow in a microfluidic channel.

36. The method of claim 34, wherein said suspension is spin-coated to said substrate at a rate between about 1000 rpm to about 5000 rpm.

37. The method of claim 30, wherein said unreacted suspension layer is removed from said substrate by dissolving said suspension layer in at least one component selected from the group consisting of: phosphate buffered saline, cell culture medium, and any combinations thereof.

38. A method of forming a three-dimensional microstructure on a substrate, said method comprising:
forming a microfluidic network comprising at least one microchannel on said substrate;
filling said microchannel with a gel precursor solution;
exposing said gel precursor to an ultraviolet light source; and
removing said microfluidic network from said substrate leaving said three-dimensional hydrogel microstructure disposed on said substrate.

39. The method of claim 38, wherein said substrate is selected from the group consisting of: glass, silicon, plastic, rubber, ceramic and any combinations thereof.

40. The method of claim 38, wherein said microchannel is formed from at least one polymeric material.

41. The method of claim 40, wherein said polymeric material is poly(dimethyl siloxane).

42. The method of claim 38, wherein said gel precursor is at least one selected from the group consisting of: poly(ethylene glycol), poly(ethylene glycol) diacrylate, photoinitiator, cell suspension, cell culture media, and any combinations thereof.

43. The method of claim 38, wherein said microfluidic network is removed from said substrate mechanically while leaving hydrogel microstructures attached to the substrate.

44. The method of claim 38, further comprising, prior to exposing said gel to said ultraviolet lights, applying a photomask over said microchannel.

45. A method of analyzing cells comprising the steps of:
forming at least one three-dimensional hydrogel microstructure having said cells encapsulated therein; and
analyzing said cells.

46. The method of claim 45, wherein said cells are mammalian cells.

47. The method of claim 45, wherein said cells are comprised of two or more phenotypes.

48. The method of claim 45, wherein said cells are comprised of mammalian cells and bacteria.

49. The method of claim 45, wherein said cells are analyzed by a monitoring means, said monitoring means is selected from the group consisting of: fluorescence including lifetime and polarization techniques, electrochemical, absorbance, chemiluminescence, surface acoustic wave mass sensors, magnetoelastic mass sensors or any combinations thereof.

50. The method of claim 45, wherein said cells are analyzed for one or more effects selected from the group consisting of: toxicity, cell morphology, apoptosis, differentiation, cell-cell interaction, cell-matrix interaction, host-pathogen interactions, endocytosis, exocytosis, and any combinations thereof.

51. A method for drug candidate screening comprising the steps of:
preparing a substrate having at least one cell-containing three-dimensional hydrogel microstructures disposed thereon;
delivering at least one reagent to said cell-containing three-dimensional hydrogel microstructure;
contacting said reagent with said cells, bacteria or mixtures thereof which are encapsulated in said cell-containing three-dimensional hydrogel microstructure; and
monitoring said cells.
